(19) United States
(12) Patent Application Publication
(10) Pub. No.: US 2008/0041749 A1
(43) Pub. Date: Feb. 21, 2008

(54) Re-closable vessel system for repeat administration of a drug product and method

(76) Inventor: Charles D. McDermott, Encinitas, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/507,175

(22) Filed: Aug. 21, 2006

(51) Int. Cl. B65D 83/00; A61K 8/84

(52) U.S. Cl. 206/438; 514/256

(57) ABSTRACT

A re-closable vessel system for repeated administration of a drug product includes a vessel docking station and an applicator along with a cyclopentane heptanoic acid, 2-cycloalkyl or arylalkyl compound represented by the formula:

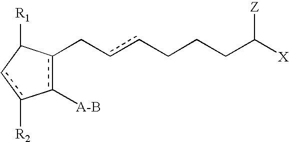

wherein the dashed bonds represent a single or double bond which can be in the cis or trans configuration, A, B, Z, X, R₁ and R₂ are as defined in the specification.

The present invention is generally related to the typical administration of drug product and is more particularly directed to a method for applying a hair growth stimulation formulation and a hair growth application system.

Applicators for the delivery of fluids to an eye have long been used for a great number of purposes. As an example, the common eye bath for comfort, refreshment, or rinsing may include a cup having an anatomically adapted design for emersion of the eye. In addition, delivery of a large fluid amounts by a spray device have also been used for eyewashes in emergency situations.

The present invention is particularly directed to applicators suitable for the administration of very small amounts of fluids to an eyelid by self-administration. In general a medical formulation should be delivered in a well defined volume to assure a specific dose to be delivered and/or absorbed. A large surplus is not desirable due to the possibility of absorbance in non-targeted tissues. This is particularly important for the application of a hair growth stimulation formulation such as set forth in U.S. patent application Ser. No. 10/345,738 filed Jan. 15, 2003 entitled “Method of Enhancing Hair Growth”. This application is to be incorporated herein in its entirety.

Minute volumes are also important with regard to price considerations for expensive medication which require frequent daily administration.

These consideration place severe demands on an applicator for administering a growth stimulation formulation for eyelashes. Small formulation amounts must be positioned with great care in order to prevent the application by overflow to non-targeted areas.

It is further and desirable that administration be conducted in various body positions and independent of applicator orientation.

In addition it is important to maintain sterility of formulation when utilized in an applicator suitable for multiple dosing.

As hereinabove noted, minute volumes are utilized and as a result vials, or vessels, or bottles containing the formulations are necessarily small. This presents an inconvenience for user handling.

To facilitate such handling, the vessel is compatible with a vanity display docking station into which the vessel may slide and be locked securely thereto. The station thereby provides the stability for facilitating the use and application of the formulation which reduces spillage due to inadvertent handling or knocking over the vessel during utilization of an applicator. The docking station may also be designed to be aesthetically pleasing and can be made from a variety of materials including but not limited to plastic, metal, or glass with normal, polished, colored, brushed, or frosted finishes.

The present invention also provides for a method of applying a hair growth stimulation formulations to mammalian skin, particularly an eyelid utilizing a system suitable for multiple dosing yet enabling sterile storage of the formulation.

### SUMMARY OF THE INVENTION

A re-closable vessel system in accordance with the present invention generally includes a vessel, a lid/applicator, a vanity docking slot, and a formulation disposed in the vessel, said formulation comprising a cyclopentane heptanoic acid, 2-cycloalkyl or arylalkyl compound represented by the formula I:

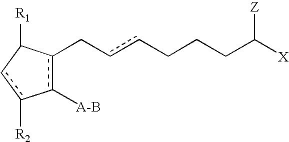

wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

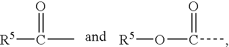

wherein R⁵ is a lower alkyl radical having from one to six carbon atoms; Z is ═O; one of R₁ and R₂ is ═O, —OH or a —O(CO)R⁶ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

Preferably, the concentration of the compound applied is from about 0.0000001% to about 50% by weight of the composition and a maximum of 3 ml of said formulation is disposed in said container.

Preferably, the compound is a cyclopentane heptanoic acid, 2-(phenyl alkyl or phenyloxyalkyl) represented by the formula II:

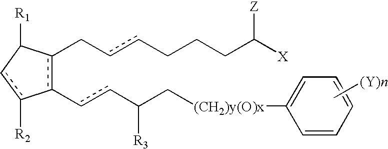

wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

More preferably the compound is a compound of formula III.

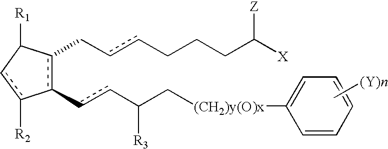

wherein hatched lines indicate α configuration, solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1-trans-pentenyl)-3,5-dihydroxy, [1_{α},2_{β},3_{α},5α], also known as bimatoprost.

The applicator device may be an applicator brush, porous foam swab or pad, hollow tube, eye dropper, dip stick, bulb or any combination thereof.

A catch mechanism is preferably provided for releasably locking the vessel and the station to one another.

A method of applying a drug product for growing eyelash includes disposing a vessel containing the formulation into a vanity display docking station, latching the vessel to the station, removing a lid, including an applicator attached thereto, and repeating applying formulation via said applicator to a user's eyelid or eyelash and subsequent to such application resealing the lid to said vessel. The applied formulation comprises a cyclopentane heptanoic acid, 2-cycloalkyl or arylalkyl compound represented by the formula:

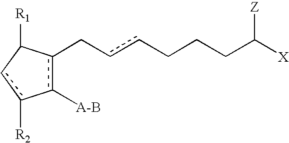

wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

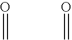

R⁵—C— and R⁵—O—C—, wherein R⁵ is a lower alkyl radical having from one to six carbon atoms; Z is ═O; one of R₁ and R₂ is ═O, —OH or a —O(CO)R⁶ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof; said formulation being packaged in a container preventing contamination of the formulation.

More particularly, the method of the present invention may include a compound of a formula:

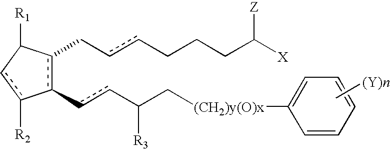

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

### BRIEF DESCRIPTION OF THE DRAWINGS

The advantages and features of the present invention will be better understood by the following description when considered in conjunction with the accompanying drawings, in which:

FIG. 1 is an exploded view of a vessel system in accordance with the present invention generally showing a removable vessel, a vanity docking station along with a catch mechanism;

FIG. 2 is a cross sectional view of the system shown in FIG. 1 illustrating a cylindrically shaped vessel;

FIG. 3 is an alternative embodiment of the present invention showing a cross section utilizing a conical vessel along with a corresponding conical cavity in a docking station along with a shoulder engaging latch for providing a catch mechanism to secure the vessel within the station;

FIG. 4 is a perspective view of a lid along with an applicator having a shank and a sponge disposed on a distal end thereof;

FIG. 5 is a lid similar to that shown in FIG. 4 utilizing a brush at a distal end of the shank;

FIG. 6 is an applicator similar to that shown in FIGS. 4 and 5 illustrating a bulb disposed at the end of an applicator shank; and

FIG. 7 illustrates a method of application formulation to a user's eyelid/eyelash.

### DETAILED DESCRIPTION

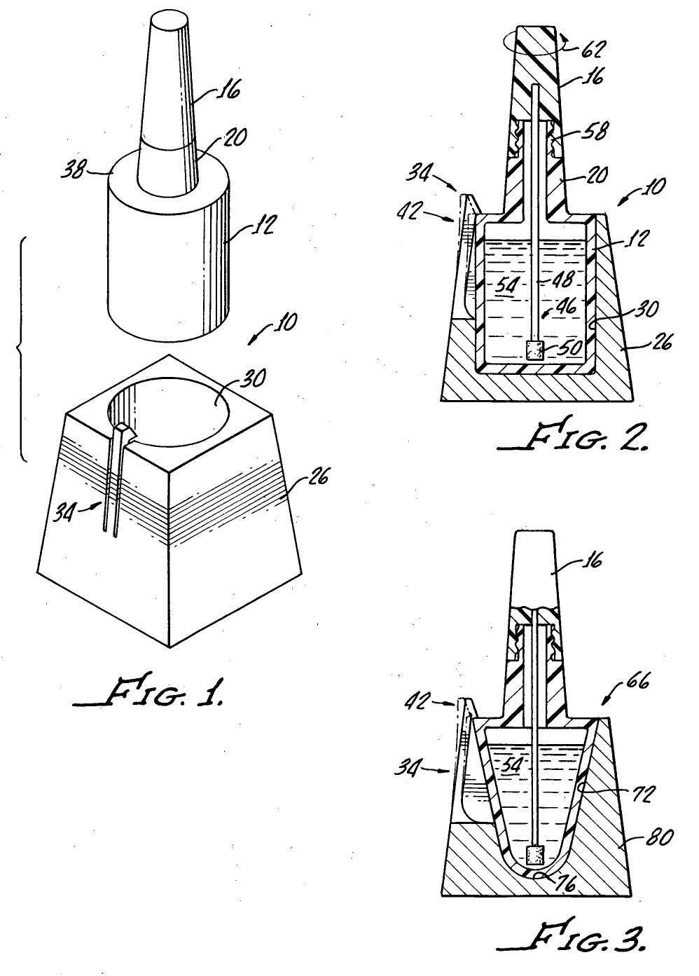

With reference to FIG. 1, there is shown a re-closable vessel system 10 in accordance with the present invention for repeated administration of a drug product which includes a vessel, which may be cylindrical, along with a removable lid 16 disposed in a sealing arrangement with a top 20 of the vessel 12 along with a vanity display docking station 26 having a cavity 30 therein sized for receiving the vessel 12 along with a catch mechanism 34 for engaging a shoulder 38 of the vessel for releasably locking the vessel 12 and the station 26 to one another.

As illustrated in FIG. 2, the catch mechanism 34 includes a movable latch 42 for releasably securing the vessel 12 within the cavity 30.

As shown in FIG. 2, the vessel system 10 includes an applicator 46 attached to the lid 16 which includes a shank 48 and a head 50 submerged in a formulation for administering the formulation to a surface, not shown.

The lid 16 preferably is in threaded 58 engagement with the top 20 in order to provide a secure seal, with opening, closing, removal and insertion of the applicator facilitated by rotating the lid 16 as indicated by the arrow 62.

The vanity display docking station 62 is preferably made from a heavy material such as, for example, glass, and of sufficient size for providing a mass sufficient to stabilize the vessel 12 upon repeated removal and insertion of the applicator 48 and lid 16 including unsealing and sealing of the lid to the vessel top.

An alternative embodiment 66 of the vessel system in accordance with the present invention is illustrated in FIG. 3 with common reference characters illustrating identical or substantially similar elements as hereinabove described in connection with the embodiment 10 shown in FIGS. 1 and 2.

The system 66 is similar to the embodiment 10, however, includes a conical vessel 72 fitted to a corresponding cavity 76 in a docking station 80.

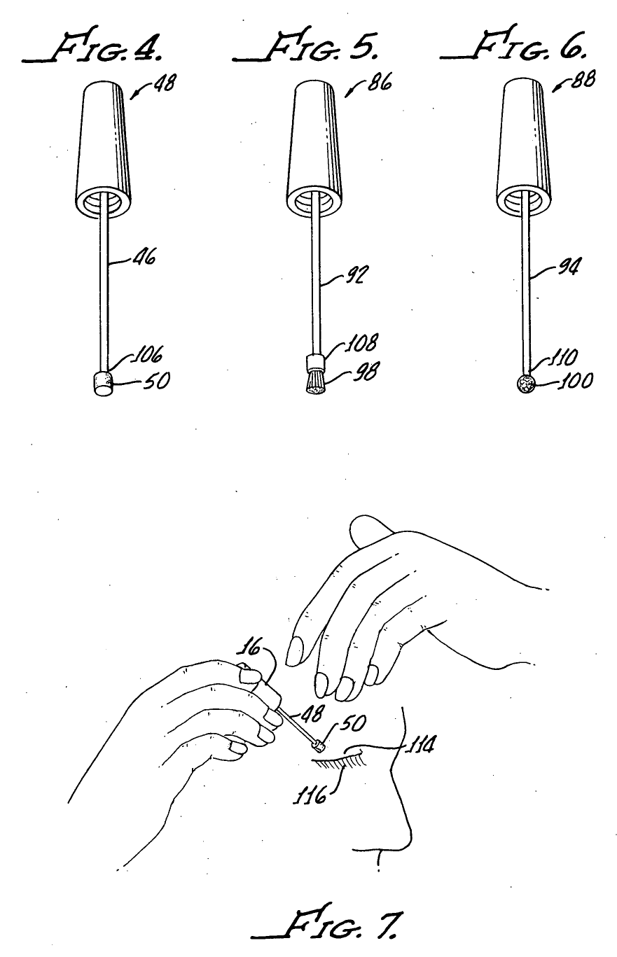

Various applicators 48, 86, 88 are illustrated in FIGS. 4, 5, and 6 each including a shank 48, 92, 94 respectively having a sponge head 50, a brush 98 or a bulb 100 disposed at a distal ends 106, 108, 110 of the shanks 48, 92, 94. The shank enabling the sponge 50, brush 98, or bulb 100 to be submerged in the formulation 54, as illustrated in FIGS. 2 and 3.

As part of the present invention, the formulation 54, disposed in the vessel 12, 72 is a cyclopentane heptanoic acid, 2-cycloalkyl or arylalkyl compound represented by the formula I:

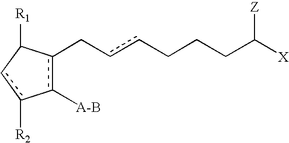

wherein dashed bonds represent a single or double bond which can be in the cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms,

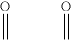

R⁵—C— and R⁵—O—C—, wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R⁶ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof. A pharmaceutical carrier adapter for topical application to mammalian skin may be provided.

Preferably, the compound is a cyclopentane heptanoic acid, 2-(phenyl alkyl or phenyloxyalkyl) presented by the formula II:

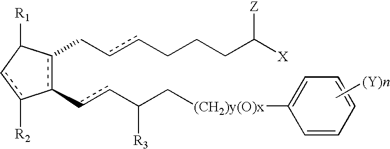

Wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

More Preferably the compound is a compound of formula III:

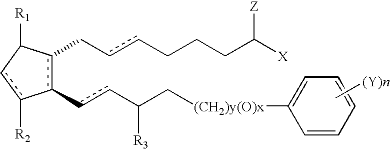

wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

More preferably y is 1 and x is 0 and R₁, R₂ and R₃ are hydroxy.

Most preferably the compound is cyclopentane N-ethyl heptanamide-5-cis-2-(3α-hydroxy-5-phenyl-1trans-pentenyl)-3,5-dihydroxy, [1_{α},2_{β},3_{α},5_{α}], also known as bimatoprost (Lumigan®).

A maximum of up to about 3 ml of the formulation 28 is disposed in the squeezable container 14, which may be formed from any suitable material to maintain sterility of the formulation. The container 14 size is selected for enabling convenient handling by a user. As indicated in FIG. 3 by arrows 38, pressure applied to the container 14 squeezes formulation through the bristles 24 as indicated by the arrow 40 in FIG. 3 to effect application of formulation 28 to the eyelid 32, eyelash 34 as shown in FIG. 2. In addition, the concentration of a compound may be from about 0.0000001% to about 50% of the composition.

After application, the brush 102 and cap 98 are again reversed for insertion into the sheath 102 as illustrated in FIG. 5. This embodiment 70 provides close control over the amount of formulation applied to the brush 100 and thereafter to the eyelid 32 which, as hereinabove noted, should be between about 20 and 50 ml.

An aqueous solution containing 5%, by weight, bimatoprost is prepared as follows. Bimatoprost is dissolved in water and the resulting solution is sterilized by filtration. The solution is aseptically filled into the sterile vessel 12, 72. Thereafter, as hereinbefore stated, the applicator is used to apply the formulation 54 to an eyelid 14 and/or eyelash 16 through the sponge 40, brush 98, or bulb 100, as illustrated in FIG. 8.

Accordingly a method in accordance with the present invention for providing a drug product, preferably for growing eyelashes, generally includes disposing a vessel 12 containing a formulation 54 into the vanity display docking station 26 and latching the vessel 12 to the station 26.

Following the latching, the method includes removing a lid 16 including an applicator 46 attached thereto with the applicator earlier submerged in the formulation 54.

The method further includes repeatedly applying formulation 54 via the applicator 46 to the user's eyelid 114, or eyelash 116 (FIG. 7) and subsequent to the application of the formulation resealing the lid 16 to the vessel 12 again submerging the applicator 46 in the formulation 54.

Further, the method includes utilization of the formulation hereinabove described.

Although there has been hereinabove described a specific re-closable vessel system and method for the repeat administration of drug product in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

1. A re-closeable vessel system for repeat administration of a drug product comprising:
a vessel;
a formulation disposed in said vessel;
a removable lid disposed in a sealing arrangement with a top of said vessel;
an applicator attached to said lid, extending into said formulation and removable with said lid from said vessel for administering said formulation to a surface;
a vanity display docking station receiving said vessel and having a mass sufficient to stabilize said vessel upon repeated removal and insertion of said applicator into formulation and sealing of the lid to the vessel top; and
a catch mechanism releasably locking the vessel and the station to one another,
said formulation comprising a cyclopentane heptanoic acid, 2-cycloalkyl or arylalkyl compound represented by the formula I: 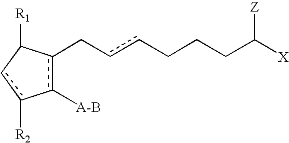
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 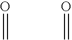
R⁵—C— and R⁵—O—C—, wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R₆ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

2. The vessel system according to claim 1 wherein the concentration of the compound applied is from about 0.0000001% to about 50% by weight of the composition.

3. The vessel system according to claim 1 wherein the compound is a cyclopentane heptanoic acid, 2-(phenyl alkyl or phenyloxyalkyl) represented by the formula II: 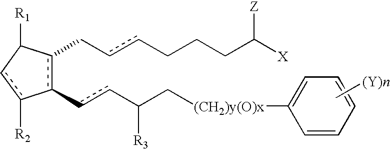
Wherein y is 0 or 1, x is 0 or 1 and x and y are not both 1, Y is a radical selected from the group consisting of alkyl, halo, e.g. fluoro, chloro, etc., nitro, amino, thiol, hydroxy, alkyloxy, alkylcarboxy, halo substituted alkyl wherein said alkyl radical comprises from one to six carbon atoms, etc. and n is 0 or an integer of from 1 to 3 and R₃ is ═O, —OH or —O(CO)R₆ wherein R₆ is as defined above or a pharmaceutically acceptable salt thereof.

4. The vessel system according to claim 1 wherein the compound is a compound of a formula III: 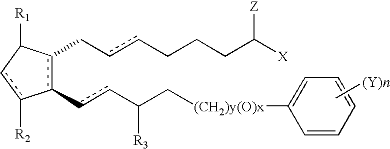
wherein y is 0 or 1, x is 0 or 1 and x and y are not both 1, Y is a radical selected from the group consisting of alkyl, halo, nitro, amino, thiol, hydroxy, alkyloxy, alkylcarboxy, halo substituted alkyl wherein said alkyl radical comprises from one to six carbon atoms, n is 0 or an integer of from 1 to about 3 and R₃ is ═O, —OH or —O(CO)R₆ wherein R₆, hatched lines indicate α configuration and solid triangles are used to indicate β configuration.

5. The vessel system according to claim 4 wherein the compound applied is bimatoprost in the form of a free base or acid addition salts thereof.

6. The vessel system according to claim 1 wherein said vessel has a generally cylindrical shape.

7. The vessel system according to claim 1 wherein said vessel has a generally conical shape.

8. The vessel system according to claim 1 wherein said applicator includes a shank with a sponge disposed at a distal end thereof.

9. The vessel system according to claim 1 wherein said applicator includes a shank with a brush disposed at a distal end thereof.

10. The vessel system according to claim 1 wherein said applicator includes a shank with a bulb disposed at a distal end thereof.

11. The vessel system according to claim 1 wherein said catch mechanism comprises a latch disposed on the station and a shoulder formed in said vessel.

12. A re-closable vessel system for repeat administration of a drug product comprises:
a plurality of vessels;
a formulation disposed in each vessel;
a plurality of removable lids disposed in a sealing arrangement with a top of a corresponding vessel;
a plurality of applicators attached to corresponding lids and extending into the formulation disposed in corresponding vessels, and removable with the corresponding lid for the corresponding vessel for administration of the formulation to a surface;
a vanity display docking station receiving the vessel one at a time and having a mass sufficient to stabilize an inserted vessel upon repeated removal and insertion of said applicator into said formulation and sealing of the corresponding lid to said inserted vessel; and
a catch mechanism releasably locking said inserted vessel and the station to one another,
said formulation comprising a cyclopentane heptanoic acid, 2-cycloalkyl or arylalkyl compound represented by the formula I: 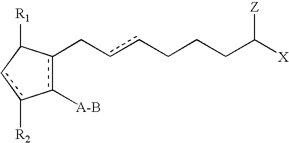
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 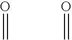
R⁵—C— and R⁵—O—C—, wherein R⁵ is a lower alkyl radical having from one to six carbon atoms; Z is ═O; one of R₁ and R₂ is ═O, —OH or a —O(CO)R⁶ group, and the other one is —OH or —O(CO)R₆, or R₁ is ═O and R₂ is H, wherein R⁶ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

13. The method of claim 12 wherein the concentration of the compound administered is from about 0.0000001% to about 50% by weight of the composition.

14. The system according to claim 12 wherein each of the vessels has a generally cylindrical shape.

15. The system according to claim 12 wherein each of the vessels has a cylindrical conical shape.

16. The system according to claim 12 wherein each applicator includes a shank with a sponge disposed at a distal end thereof.

17. The system according to claim 12 wherein each applicator includes a shank with a brush disposed at a distal end thereof.

18. The system according to claim 12 wherein each applicator includes a shank with a bulb disposed at a distal end thereof.

19. The system according to claim 12 wherein said catch mechanism comprises a latch disposed on the station and a shoulder formed in each of the vessels

20. A method for applying a drug product for growing eyelashes, said method comprising:
disposing a vessel containing a formulation into a vanity display docking station;
latching said vessel to the station;
removing a lid including an applicator attached thereto, said applicator being submerged in said formulation;
repeatedly applying said formulation via said applicator to a user's eyelash; and
subsequent to application of said formula into the user's eyelash, resealing said lid to said vessel with the applicator submerged in said formula,
said formulation comprising a cyclopentane heptanoic acid, 2-cycloalkyl or arylalkyl compound represented by the formula I: 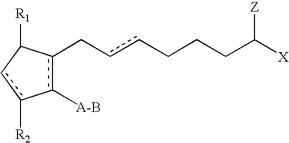
wherein dashed bonds represent a single or double bond which can be in a cis or trans configuration, A is an alkylene or alkenylene radical having from two to six carbon atoms, which radical may be interrupted by one or more oxa radicals and substituted with one or more hydroxy, oxo, alkyloxy or akylcarboxy groups wherein said alkyl radical comprises from one to six carbon atoms; B is a cycloalkyl radical having from three to seven carbon atoms, or an aryl radical, selected from the group consisting of hydrocarbyl aryl and heteroaryl radicals having from four to ten carbon atoms wherein the heteroatom is selected from the group consisting of nitrogen, oxygen and sulfur atoms; X is —N(R⁴)₂ wherein R⁴ is selected from the group consisting of hydrogen, a lower alkyl radical having from one to six carbon atoms, 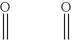
R⁵—C— and R⁵—O—C—, wherein R⁵ is a lower alkyl radical having from one to six carbon atoms; Z is ═O; one of R₁ and R₂ is ═O, —OH or a —O(CO)R₆ group, and the other one is —OH or —O(CO)R₆, or R₁ is ═O and R₂ is H, wherein R⁶ is a saturated or unsaturated acyclic hydrocarbon group having from 1 to about 20 carbon atoms, or —(CH₂)ₘR₇ wherein m is 0 or an integer of from 1 to 10, and R₇ is cycloalkyl radical, having from three to seven carbon atoms, or a hydrocarbyl aryl or heteroaryl radical, as defined above or a pharmacologically acceptable acid addition salt thereof.

21. The method according to claim 20 wherein the concentration of the compound administered is from about 0.0000001% to about 50% by weight of the composition.

22. The method according to claim 20 further comprises releasing said vessel from the station following administration of all the formulation from the vessel.
